(19) United States
(12) Patent Application Publication
(10) Pub. No.: US 2001/0051825 A1
(43) Pub. Date: Dec. 13, 2001

(54) Temporary lenses and method for providing correct lens power

(76) Inventor: Norman D. Peterson, Laguna Hills, CA (US)

Correspondence Address: Boniard I. Brown, 1500 West Covina Parkway, #113, West Covina, CA, 91790-2793, US

(21) Appl. No.: 09/932,493

(22) Filed: Aug. 20, 2001

Related U.S. Application Data
(63) Continuation-in-part of application No. 09/472,306, filed on Dec. 27, 1999.

(51) Int. Cl. A61F 2/16

(52) U.S. Cl. 623/6.12; 623/6.43; 623/912

(57) ABSTRACT

Temporary intraocular lenses are provided for disposing in eyes after removal of natural lenses. According to the invention, retinoscopy is performed with one of successive lenses to determine appropriate optical power of a permanent lens which is then installed. Temporary economical lenses are foldable and adapted for such procedures.

### RELATED APPLICATIONS

This application is a continuation-in-part of my co-pending application Ser. No. 09/472,306, filed Dec. 27, 1999.

### BACKGROUND AND SUMMARY OF THE INVENTION

The present invention relates generally to ophthalmology, and particularly to the utilization of a temporary intraocular lens for determining the optical power requirement for a permanent intraocular lens to be implanted in a particular eye, whereafter the temporary lens is removed and a permanent lens of predetermined optical power is installed.

The present invention provides substantial assurance that a permanent lens of correct optical power is implanted in a first surgical procedure. FIG. 5 shows a preferred form of intraocular lens 30 disposed in an eye and comprising an optic 32 and oppositely extending haptics 34, 36 engaged in a capsular bag rim.

It is desired to avoid the necessity of removal of an implanted lens after installation thereof in an eye, then replace it with another lens of more accurate optical characteristics for a particular eye.

With an unsuitable intraocular lens implanted with an unsatisfactory outcome for the patient, it may result in optical imbalance between the two eyes of a patient, substantial patient dissatisfaction, increased dependence upon optical lenses or contact lenses, and additional surgery. Such additional surgery involves substantial cost and time and effort of the surgeon, patient inconvenience, suffering and trauma, medical risks, and legal complications.

A variety of causes may result in error in the final outcome of such surgery, utilizing established techniques. The length of the particular eye may not be accurately measured, or corneal curvature may not be accurately measured, or the corneal-retinal distance may not be accurately pre-operatively measured by ultrasound, etc.

Accommodating lenses, these being adjustable focus lenses, require precision in power selection; replacement of such lenses is inherently more costly and more complex and likely to result in probably requiring further surgical intervention. The present invention may be utilized with various types of intraocular lenses, and relative to various intraocular lens procedures.

The invention provides temporary evaluation lenses and methods of using the same, to enable the provision of an intraocular lens of correct power for insertion into an eye of a particular person. The temporary lens has an optical power estimated for an appropriate permanent lens. With the temporary lens disposed in the eye, retinoscopy is performed and the temporary lens is then removed, and a correct permanent intraocular lens is implanted, this lens having a power determined by the retinoscopy. A preferred evaluation lens is provided which is of simplified and economical structure.

FIG. 4 illustrates the disposition of a temporary IOL in an eye in more than one position. As shown, it may be disposed in the anterior chamber of the eye on the anterior surface of the iris, or within the capsular bag remnant after the natural lens is extracted, or between the capsular bag and the posterior surface of the iris. Each such different location of the temporary IOL action results in a different optical power result for a given lens.

It is desirable that a temporary lens be relatively easily inserted into an eye and removed therefrom, without substantial detailed procedure. A temporary lens is selected for having optical power generally approximating the estimated optical power needed in a permanent implanted lens.

It may be appropriate or necessary to insert and remove successive temporary lenses determining the accurate optical power for the permanent intraocular lens. Often, a surgeon may interpose a succession of lens of different respective powers in order to neutralize motion during retinoscopy in order to determine the correct desired optical power of the permanent IOL. A temporary IOL may be smaller in size than a permanent IOL. An optic size of 4½ mm provides the needed reading to determine appropriate optical power. With the temporary lens, there is no concern regarding providing long-term optical power, but it is only necessary to provide accuracy of a central portion of a lens.

A temporary lens may be considered to be somewhat in the nature of an instrument, and more of a guide than an implant. Thus, relative ease of insertion and removal are provided, and control is maintained over the IOL positions at all times during surgical procedures. When it has been inserted in an eye, the surgeon can still control it from outside the eye, and it is removed like an instrument.

### BRIEF DESCRIPTION OF THE DRAWINGS

FIG. 1 is an exploded view diagrammatically illustrating utilization of retinoscopy for determination of correct lens power, according to the invention;

FIG. 2 is a perspective view of a temporary intraocular lens utilized in accordance with the invention;

FIG. 3 shows a sectional view of an eye and an insertion instrument during insertion of the lens of FIG. 2;

FIG. 4 shows respective positions wherein the temporary lens of FIG. 2 may be installed in an eye;

FIG. 5 shows a preferred form of intraocular lens disposed in an eye, and having an optic with oppositely extending haptics engaging a capsular bag;

FIGS. 6A and 6B show a temporary intraocular lens having three stems extending therefrom with openings to receive an instrument;

FIGS. 7A and 7B show a temporary intraocular lens having four stems extending radially therefrom with openings to receive a hook of an instrument, as shown;

FIGS. 8 and 9 are sectional views showing the temporary intraocular lens of FIGS. 7A and 7B with stems thereof disposed relative to the iris and capsular bag rim remnant;

FIGS. 10A and 10B show a temporary intraocular lens having three tapered stems extending radially therefrom, and an elongated handle extending therefrom having five openings to receive an instrument;

FIGS. 11A and 11B show a temporary intraocular lens having three spaced-apart stems extending radially therefrom extending upwardly and outwardly from an optic, and further showing a fenestration in the periphery of the optic to receive an instrument;

FIGS. 12A and 12B show a temporary intraocular lens having three stems extending radially therefrom and a suture anchor extending from the optic to receive a suture wound thereabout;

FIGS. 13A and 13B show a temporary intraocular lens having three tapered stems extending therefrom, and a suture anchor defined by notches in the periphery of the optic;

FIGS. 14A and 14B show a temporary intraocular lens having three stems extending radially therefrom and having openings therein, one of the stems extending upwardly and outwardly from a periphery of the optic;

FIGS. 15A and 15B show a temporary intraocular lens having three tapered stems extending radially therefrom, and an elongated handle extending upwardly and outwardly from the optic with openings therein; and

FIGS. 16A and 16B show a temporary intraocular lens having three stems extending radially therefrom and an instrument retainer defined in a peripheral portion of the optic.

### DESCRIPTION OF THE PREFERRED EMBODIMENTS

The present invention utilizes a foldable temporary intraocular lens which is inserted into an eye for evaluation by a surgeon of the refractive status of the eye relative to the optical power of the temporary lens. The temporary lens serves to evaluate the optical function of the eye to determine the appropriate and correct power for a permanent lens implant.

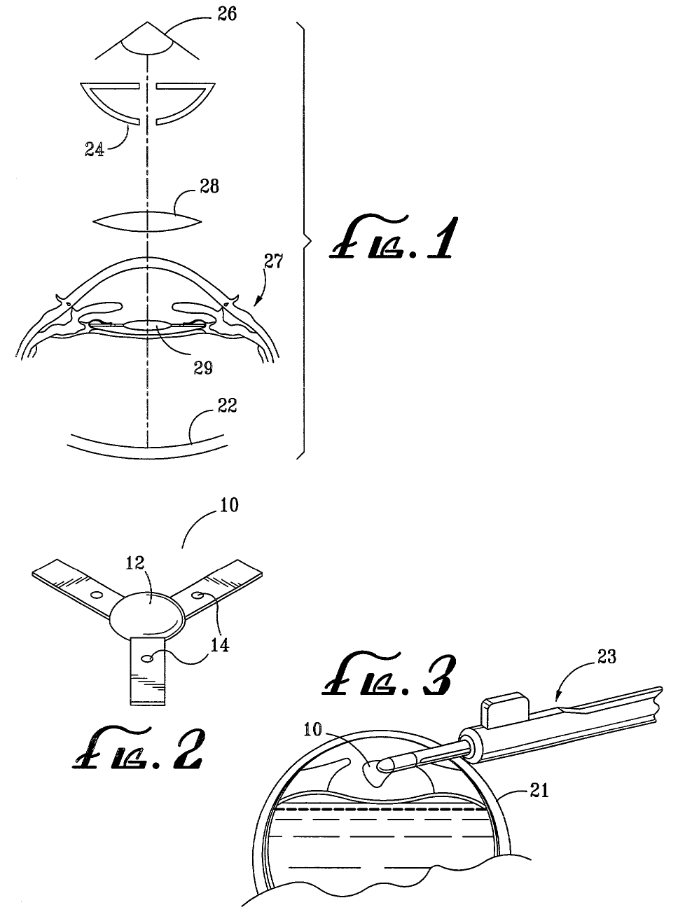

Referring to the drawings, particularly FIGS. 1 and 2, a temporary lens 10 is shown as comprising a central optic 12, and three haptics 14, attached to and extending radially therefrom. This lens has an optic selected to approximate the estimated power of the permanent lens to be inserted. The temporary lens is flexible, foldable, and is implantable via a small incision in the eye, by known procedures, or by other methods. The temporary lens is inexpensive and disposable after use.

The temporary lens 10 is utilized with the present invention to provide data, by retinoscopy, etc., to enable the provision of a permanent intraocular lens of correct power and accuracy into the eye of a person.

The temporary lens 10 has three haptics 14 extending at 120° intervals about the optic 12, the outer edges of the haptic falling in a circle of about 12.75 mm diameter.

Perforations of about 0.5 mm diameter are disposed on the haptics 14 to better enable handling and manipulation by surgical instrumentation, and for removal from an eye by an instrument.

Utilizing techniques known in the art, the temporary lens 10 may be positioned, as shown in FIG. 4, anteriorly of an iris, as indicated at 16, posteriorly of the iris, indicated at 18, or within the capsular bag, as indicated at 20. As indicated in FIG. 3, insertion of lens 10 via a slit in the cornea 21 is by an insertion instrument 23.

Referring to FIG. 1, at the time of surgery, retinoscopy is utilized to determine the effectiveness of a selected lens optic, and to quantify any adjustment necessary in the power of the intraocular lens for final insertion. By retinoscopy, well known in the art, reflections from the retina 22 of the eye 27 are utilized involving a light source 24 and the eye 26 of the surgeon to determine the total optical power of an eye having a natural lens 29. By interposing a succession of temporary lenses 28, the surgeon can calculate any required additional lens power required for desired optical power in a particular case.

Following retinoscopy, the temporary lens 10 is extracted from the eye by appropriate instrumentation and known techniques, the lens passing from the eye via a small incision.

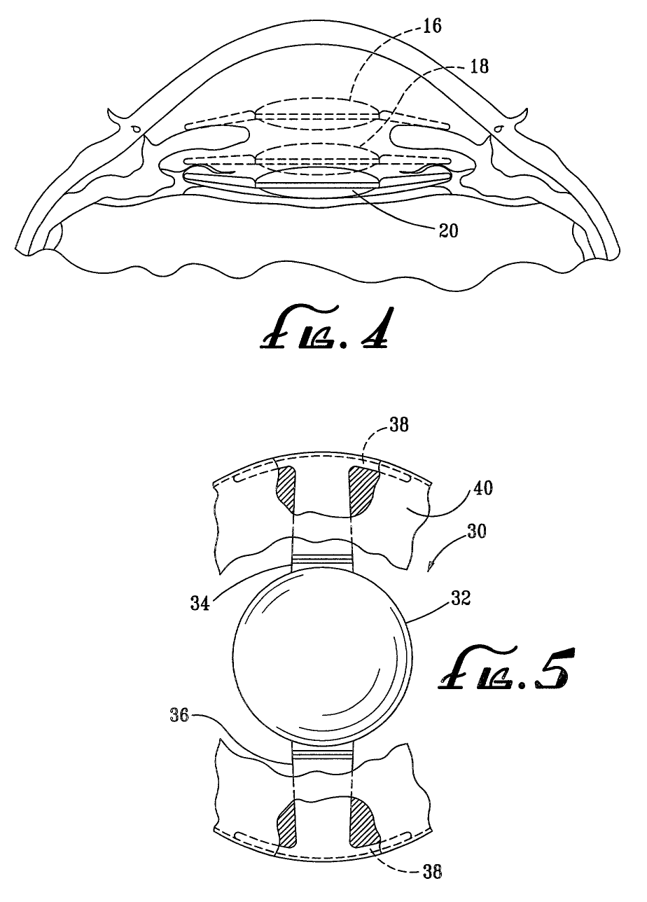

Utilizing information derived from retinoscopy and utilization of the temporary lens, the surgeon determines the power of a permanent intraocular lens 30 (FIG. 5) which is inserted by known techniques for desired optical results. The lens 30 comprises an optic 32 and haptics 34, 36 extending oppositely therefrom to engage opposite portions of a capsular bag 40 equator, with wing portions 38 thereof further engaging the bag equator.

Referring to the drawings, various embodiments of temporary intraocular lenses according to the invention, are shown in FIGS. 6-16.

Each embodiment shown has at least one element which extends from an optic to enable a surgeon to manipulate the lens in the eye. The element may be a stem which does not extend outside the eye, with the lens within the eye. A handle is an element of such length that it has a portion extending outside the eye, with the optic inside the eye.

One or more openings or holes may be provided in a stem or in a handle for engagement therein of a hook portion of an instrument for the manipulation of the IOL in the eye for insertion, removal, and manipulation of the lens.

It is of course not desirable to provide holes or openings in a permanent IOL implant, because it is necessary to avoid light being disposed in any unplanned manner on any relatively permanent basis.

FIGS. 6A and 6B show a temporary IOL 50 having three stems 52, 54, 56 extending radially from an optic, and a fenestration 58 in the periphery of the optic, all of which are adapted to receive a hook or like portion of an instrument for manipulation of the optic in the eye.

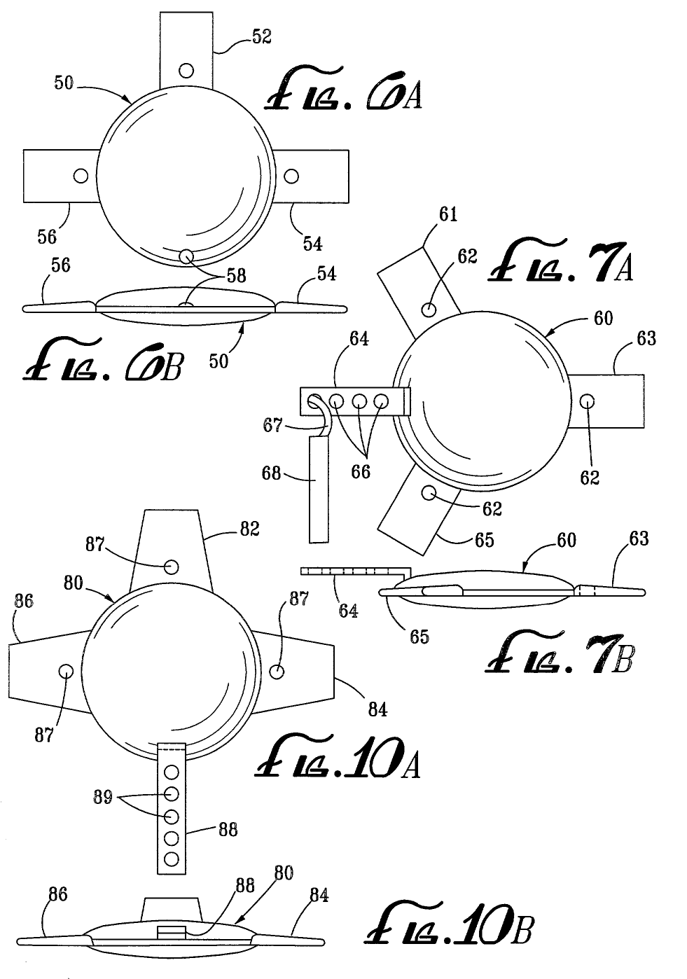

FIGS. 7A, 7B show a temporary IOL 60 with four radially-spaced stems 61, 63, 64, 65 extending from an optic in radially-spaced relation, three of such stems having a single opening 62 therein, and one stem 64 having four spaced-apart openings 66 to receive a hook portion 67 of an instrument 68, as shown. One stem 64 is relatively thick and rigid. The stem having four openings 66 therein has a portion extending upwardly or axially of the optic from a peripheral portion of the optic, and an elongated portion extending from said portion and generally perpendicular to a central axis of the optic.

FIGS. 8 and 9 show a temporary lens 70 of FIGS. 7A, 7B disposed in an eye with its stem 72 (FIG. 8) disposed on the anterior surface of the iris, with the opposite stem 74 disposed in the sulcus between the iris and the anterior capsular bag rim. FIG. 9 shows the lens 70 disposed in the eye with its left stem disposed on the anterior surface of the iris, and its opposite stem 74 disposed within the capsular bag rim 76.

Thus, a temporary IOL may be disposed in various features within an eye and in stable position during surgical procedures.

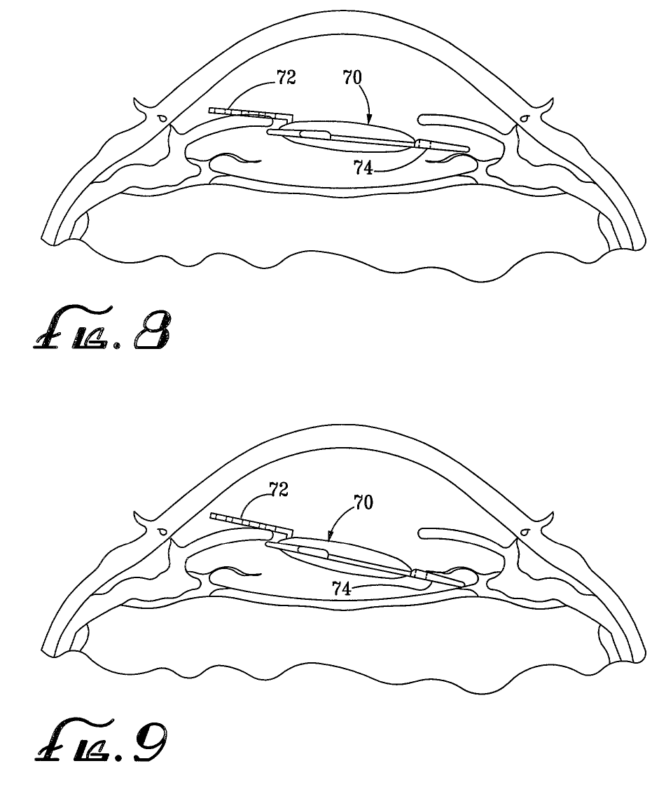

FIGS. 10A and 10B show a temporary lens 80 having three spaced-apart stems 82, 84, 86 extending outwardly from an optic and narrowing outwardly, each having an opening 87 to receive an instrument. An elongated handle 88 extends outside the eye and has five openings 89 therein for selective positioning of a hook of an instrument for manipulation of the lens within the eye.

FIGS. 11A and 11B show a temporary lens 90 with three stems 92, 94, 96 extending generally radially from an optic, each stem having a portion 98 extending upwardly from a peripheral to portion of an optic, and a portion 100 extending generally radially outwardly therefrom for positioning on a component of an eye during a surgical procedure. A fenestration 102, similar to that of FIG. 6A, is defined in a peripheral portion of the optic for insertion of an instrument to manipulate a lens within the eye.

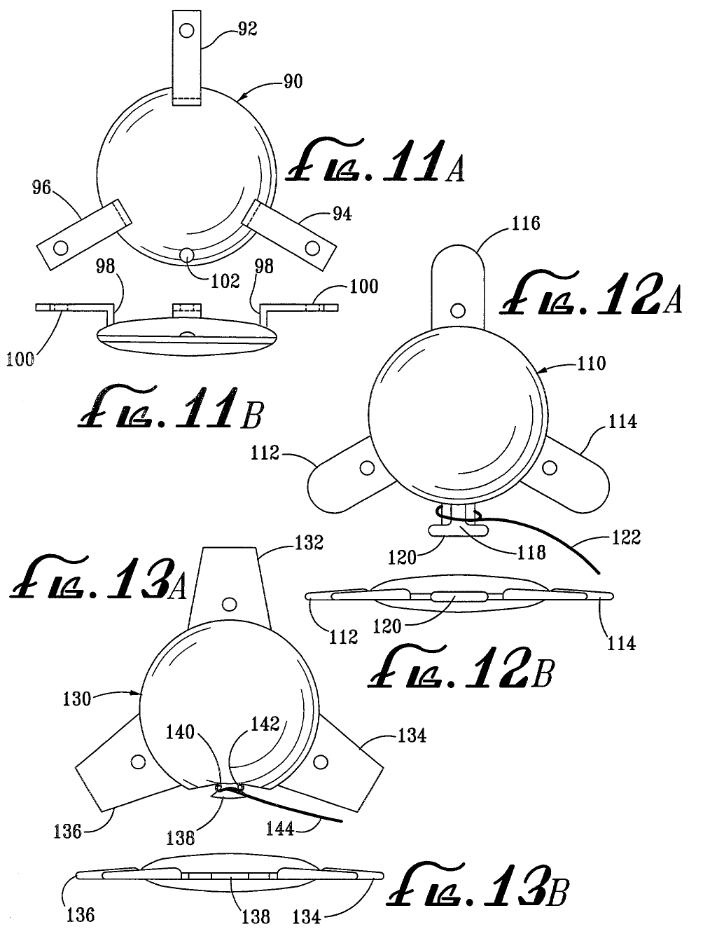

FIGS. 12A and 1212B show a lens 110 having an optic having three stems 112, 114, 116 extending generally radially from an optic. An anchor 118 extends from the optic and has a retaining shoulder 120, whereby a suture 122 may be wound on and retained by shoulder 120 of the anchor.

FIGS. 13A and 13B show a lens 130 having thee tapered generally rectilinear stems 132, 134, 136, each having an opening therein. An anchor 138 is defined in a peripheral portion of the optic by two notches 140, 142 in the optic which taper inwardly and toward each other, as shown. A suture 144 may be wound about anchor 138 and extend therefrom for manipulation by a surgeon.

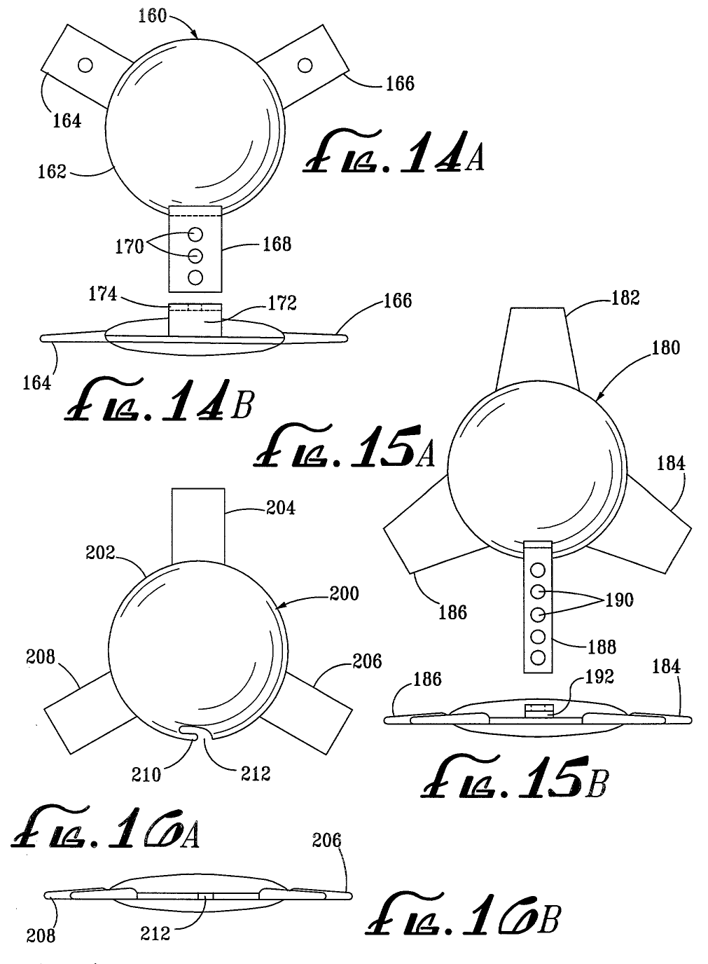

FIGS. 14A and 14B show a lens 160 having an optic 162 having extending therefrom two stems 164, 166, each having an opening therein for engagement by an instrument, and stem 168 having three openings 170 therein, and having a portion 172 extending generally parallel to an axis of the optic and another portion 174 extending from the end thereof and generally normal to the axis of the optic, similar to the stem 64 of the embodiment of FIG. 7A.

FIGS. 15A, 15B show a lens 180 having three radially extending stems 183, 184, 186, and a handle 188, with five spaced-apart openings 190, as shown. A portion 192 of the handle extends from the periphery of the optic and parallel to the axis thereof, and a second portion extends therefrom generally normal to the axis of the optic, thus to enable positioning thereof on a component of an eye in stable position during surgical procedures.

FIGS. 16A, 16B show an embodiment 200 wherein an optic 201 has three stems 204, 206, 208 extending therefrom. A retainer 210 is defined in a peripheral portion of the optic by an opening 212 defined therein, as shown. The retainer is adapted to receive an instrument hook for manipulating the optic in the eye. This structure enables accurate, limited movement of the optic in the eye for relatively precise positioning, there being no outward extension of any member to effect any substantial leverage.

It will be understood that various changes and modifications may be made from the preferred embodiments discussed above without departing from the scope of the present invention, which is established by the following claims and equivalents thereof.

The inventor claims:

1. A temporary evaluation intraocular lens for insertion into an eye of a patient to determine the optical characteristics for a permanent intraocular lens for said eye, said lens comprising:
an optic approximating the range of optical power for a permanent intraocular lens for said eye, and
at least one element extending outwardly from said optic for manual manipulation of the optic within the eye.

2. A temporary lens according to claim 1, wherein:
said element is a stem extending from the optic and within the eye.

3. A temporary lens according to claim 1, wherein:
said element comprises a handle extending from the optic and exteriorly of the eye when the optic is within the eye.

4. A temporary lens according to claim 3, wherein said handle extends radially of the optic.

5. A lens according to claim 2, wherein said element is of generally flat configuration.

6. A lens according to claim 1, wherein said element is formed by molding integrally with the optic.

7. A lens according to claim 6, wherein the lens is formed of silicone.

8. A lens according to claim 1, wherein:
said at least one element has at least one opening defined therein to receive an instrument for external manipulation of the optic in the eye.

9. A lens according to claim 8, wherein said instrument comprises a hook to engage in said element opening.

10. A lens according to claim 2, wherein:
said at least one element has at least one opening defined therein to receive an instrument for external manipulation of the optic in the eye.

11. A lens according to claim 3, wherein:
said at least one element has at least one opening defined therein to receive an instrument for external manipulation of the optic in the eye.

12. A lens according to claim 1, wherein:
a plurality of stems extend from the optic, and at least one of the stems has an opening to receive an instrument for manipulation of the lens in the eye.

13. A lens according to claim 12, wherein at least some of said stems extend generally radially from the optic in spaced relation.

14. A temporary evaluation intraocular lens for insertion into an eye of a patient to determine the optical characteristics for a permanent intraocular lens for said eye, said lens comprising:
an optic approximating the range of optical power for a permanent intraocular lens for said eye, and
a first element extending outwardly from said optic for manual manipulation of the optic within the eye,
said first element comprising a first portion extending from the periphery of the optic generally parallel to the axis of the optic, and a second portion extending from the first portion generally perpendicular to the axis of the optic to extend generally radially outwardly from the optic, and
a second element extending from the optic extending generally oppositely from the at least one element,
whereby during surgical procedure with the lens in the eye of a patient, the first element may be supported on the iris of the eye with the second element disposed in one of (a) the sulcus between the iris, (b) a capsular bag remnant rim.

15. A temporary lens according to claim 2, wherein said stem has a first portion extending from the periphery of the optic generally parallel to the axis of the optic, and a second portion extending from the first portion and perpendicular to the axis of the optic to extend outwardly from the optic,
whereby said stem is adapted to be disposed on one of (a) the iris, and (b) between the iris and capsular bag temporarily during surgery during procedure involving replacement of the lens in the eye.

16. A lens according to claim 1, wherein:
a fenestration is defined in a peripheral portion of the optic for manipulation of the lens in the eye by an instrument.

17. A lens according to claim 1, and further comprising:
a suture anchor at the periphery of the optic to receive thereabout a suture to provide a temporary anchor for a suture during surgical procedure.

18. A lens according to claim 17, wherein said suture anchor has a shoulder thereon to retain the suture.

19. A lens according to claim 17, wherein:
the suture anchor is defined between a pair of spaced-apart notches extending inwardly toward each other.

20. A lens according to claim 1, and further comprising:
a retainer arm defined in the periphery of the optic and adapted to receive an instrument for insertion, removal and manipulation during surgical procedure,
said retainer arm position in the optic periphery enabling accurate manipulation of the optic by the instrument.

21. A method of providing an intraocular lens of correct power for insertion into an eye of a particular person, comprising:
providing a temporary evaluation lens having an optical power estimated to approximate the power of an appropriate lens,
disposing said temporary lens in said eye,
performing a retinoscopy with said temporary lens positioned for determination of the refractive status of the eye,
removing said temporary lens from the eye,
providing a permanent intraocular lens having a power determined by said retinoscopy, and
installing said permanent intraocular lens in said eye.

22. A method according to claim 21, wherein:
said retinoscopy utilizes reflections from the retina of said eye to determine the optical power of the eye by interposing successive lenses in the light path to the retina of the eye.

23. A method according to claim 21, wherein said retinoscopy procedure comprises disposing successive test lenses in a retinoscopy light path.

24. A method according to claim 21, wherein said temporary lens is installed in said eye in a position which is one of (a) posteriorly of the cornea and anterior of the iris, (b) anteriorly of the capsular bag, (c) within the capsular bag.
